(19) United States
(12) Patent Application Publication
(10) Pub. No.: US 2022/0296506 A1
(43) Pub. Date: Sep. 22, 2022

(54) A METHOD FOR OBTAINING ION-EXCHANGE POLYMERIC HYDROGELS FOR EYE TREATMENT AND HYDROGEL LENSES THEREOF

(71) Applicant: SIA MEDHYDROGEL, Riga (LV)

(72) Inventors: Anastasija DREBOTE, Riga (LV); Tatsiana CHUDUK, Minsk (BY); Sergey VINOGRADOV, Minsk (BY)

(73) Assignee: SIA MEDHYDROGEL, Riga (LV)

(21) Appl. No.: 17/637,374

(22) PCT Filed: Sep. 30, 2019
(86) PCT No.: PCT/IB2019/057303

§ 371 Date: Feb. 22, 2022

(51) Int. Cl. A61K 9/00; C08F 226/08; C08J 5/22; A61K 31/185; A61K 31/728; A61K 9/51

(52) U.S. Cl. CPC A61K 9/0051; C08F 226/08; C08J 5/2231; A61K 31/185; A61K 31/728; A61K 9/5161; C08J 2339/06; C08J 2405/08

(57) ABSTRACT

The present invention could be used in medicine and relates to a method of producing ion-exchange polymeric hydrogels for eye treatment, which includes monomer copolymerization under ionizing radiation in presence of linking agent and ionites, when copolymerization is carried out gradually to obtain a prepolymer of desired viscosity for filling lens forms, and ionites introduction in the form of finely dispersed powder, filing the hydrogel into lens forms and subsequent copolymerization till an adequate ionizing dosage is performed providing a gel suitable for lenses formation, characterized in that the gel is also filled with pharmaceutically active agent in the form of finely dispersed powder prior to ionite introduction, and the size of particles of PAA is lower that the size of ionite particles. The present invention also relates to therapeutic hydrogel lenses produced in accordance with the above-mentioned method.

### TECHNICAL FIELD

An invention relates to the technology of preparation of therapeutic eye lens and particular to a method of synthesis of polymeric hydrogels that could be used in medicine for eye treatment, especially chemical burns injuries.

### BACKGROUND ART

The traditional use of drugs for eye diseases treatment in the formulation of drops or ointments is well known, but ineffective since a significant part of the drug is washed off by tears.

The use of therapeutic contact lenses for treating eye diseases using controlled delivery of drugs or pharmaceutically active substances is known in the art. This approach allows to control the flow of pharmaceutically active agents (PAA) into the eye and, when the lens contacts the surface of the eye directly, significantly reduces leaching. In addition, it is possible to carry out prolonged treatment (Seung Woo Choi, Jaeyun Kim. Therapeutic Contact Lenses with Polymeric Vehicles for Ocular Drug Delivery: A Review, 2018).

There are several known methods for preparation of lens with drug delivering function, for example:

The method of saturation of the lens gel in the drug solution (most commonly, the drug is quickly washed out) JP2001516713A Whereing eye surface or eye loaded contact lenses, compositions and methods for treating an aqueous composition comprising the quaternary nitrogen-containing ethoxylated glycoside and therapeutic agent are disclosed. The present invention is particularly suitable for use in silicone-containing contact lenses. Examples of specific compositions comprise lauryl methyl-glucamine Seto-10 hydroxypropyl dimonium chloride aqueous solution, such combination with an anionic polysaccharide as hyaluronic acid.

The method of drug administration during gel polymerization (allows you to calculate the amount, speed and duration of drug intake) EP0763754B1 Photocured crosslinked-hyaluronic acid contact lens. A contact lens having a water content of 80 to 99% by wt., and shape compatibility and tissue affinity for an eyeball, the contact lens comprising a photocured crosslinked-hyaluronic acid derivative produced by the formation of a crosslinked cyclobutane ring by light irradiation from mutual photoreactive crosslinking groups linked to carboxyl groups of hyaluronic acid via a bridging moiety.

A process for preparing a photocured crosslinked-hyaluronic acid contact lens as defined in claim 1 which comprises molding a photoreactive hyaluronic acid derivative comprising photoreactive crosslinking groups linked to carboxyl groups of hyaluronic acid via a bridging moiety into a shape that fits to the eyeball, and sub-sequently irradiating the shaped product with light to effect crosslinking by formation of a cyclobutane ring between the mutual photoreactive crosslinking groups.

These therapeutic lenses are mainly used for prolonged treatment of eye diseases; however, they cannot be used to treat severe chemical burns and other eye injuries where urgent removal of a chemical or toxic substance is required, especially in cases of deep lesions.

The disadvantages of therapeutic lenses known from the prior art are their inability to quickly absorb aggressive chemical agents that cause chemical burns to the eyes, as well as decay products resulting from damage of eye tissues. Thus, therapeutic lenses known from the prior art cannot be used to treat acute conditions caused by membrane damaging of the eye and in need of rapid relief of pain and removal of the damaging agent.

For the treatment of chemical burns, lenses containing ion exchange resins (IER) are used.

Such lenses are obtained by introducing ion exchangers in solid finely divided form into a prepolymer and subsequent polymerization by irradiation in the presence of a cross linking agent (RU patent 2 428 988).

In this method of producing ion-exchange polymer hydrogels for treating chemical burns of the eyes, the monomers are copolymerized in the presence of a cross linking agent and a mixture of equal amounts of the sodium and hydrogen forms of the ion-exchange resin and the copolymerization is carried out in the presence of a cross linking agent under the ionizing gamma radiation until an absorbed dose of 3.5-4.5 Mrad is achieved.

Moreover, a method for introducing an ion exchange resin into a polymer matrix at the gelation stage is provided. This technique allows to prevent sticking of the resin particles during the mixing process and to achieve its uniform distribution in the volume of the product.

Upon reaching an absorbed dose of 0.5 Mrad, a gel of a certain viscosity is formed, sufficient to prevent the ion-exchange resin from settling in the volume of the polymerization mold. The dried and sieved cation exchanger in sodium or hydrogen form, or a mixture of the sodium and hydrogen form of cation exchanger are added to the gel, they are packaged to the molds, and further polymerization is carried out to a total absorbed dose of 3.5-4.5 Mrad.

The resulting solid polymer in the form of a lens is hydrated, then rinsed in water to achieve a pH of 6-7, placed in bottles with distilled water or physiological saline, and sterilized by the radiation method.

This method is the closest to the claimed invention.

### SUMMARY OF INVENTION

A method of producing ion-exchange polymeric hydrogels for eye treatment, which includes monomer copolymerization under ionizing radiation in presence of linking agent and ionites, when copolymerization is carried out gradually to obtain a prepolymer of desired viscosity for filling lens forms, and ionites introduction in the form of finely dispersed powder, filing the hydrogel into lens forms and subsequent copolymerization till an adequate ionizing dosage is performed providing a gel suitable for lenses formation, characterized in that the gel is also filled with pharmaceutically active agent in the form of finely dispersed powder prior to ionite introduction, and the size of particles of pharmaceutically active agent is lower that the size of ionite particles.

The method of producing ion-exchange polymer hydrogels, characterized in that at the prepolymer stage (during process) microporous sorbents selected from the group consisting of silica gel, salts of alginic acids, carbon sorbents, zeolites in the form of fine powders are additionally introduced.

The method of producing ion-exchange polymer hydrogels, characterized in that pharmaceutically active agent is hyaluronic acid in the form of solid finely dispersed powder.

The method of producing ion-exchange polymer hydrogels, characterized in that hyaluronic acid under some types of ionizing radiation breaks down into low-molecular-weight hyaluronic acid, which possesses high ability for wound healing.

The method of producing ion-exchange polymer hydrogels, characterized in that pharmaceutically active agent is taurine.

The method of producing ion-exchange polymer hydrogels, characterized in that pharmaceutically active agent represented by nanoparticles with antimicrobial function.

The method of producing ion-exchange hydrogels, characterized in that pharmaceutically active agent are introduced in capsules having a shell with a delayed pharmaceutically active agent release.

The method of producing ion-exchange hydrogels, characterized in that the capsules have a shell consisting from alginic acid salts or chitosan.

The method of producing ion-exchange hydrogels, characterized in that the pharmaceutically active agent introduced in capsules selected from a group consisting of taurine, retinol, cholecalciferol, tocopherol, lutein, zeaxanthin, flavonoids, antibiotics, anti-inflammatory and analgesic agents.

The method of producing ion-exchange hydrogels, characterized in that above-mentioned capsules also includes nanoparticles or accelerated penetration of pharmaceutically active agent throw ocular barriers.

The method of producing ion-exchange hydrogels, characterized in that nanoparticles included into capsules with pharmaceutically active agent, are antiferromagnetic or superparamagnetic nanoparticles for managed activation of pharmaceutically active agent release.

Therapeutic hydrogel contact lenses with ionites, produced from hydrogel in accordance method, wherein the mentioned above hydrogel includes bound and distributed particles of pharmaceutically active agent with sizes less that ionite particles size.

Lenses, characterized in that particles are capsules with pharmaceutically active agent.

Lenses, characterized in that pharmaceutically active agent is hyaluronic acid.

Lenses, characterized in that particles are capsules with pharmaceutically active agent have a shell with delayed pharmaceutically active agent release.

Lenses, characterized in that capsules have shell produced from alginates.

Lenses, characterized in that pharmaceutically active agent particle size range from 50 to 1000 nm.

Lenses, characterized in that pharmaceutically active agent particles includes magnetic and/or superparamagnetic nanoparticles.

### Technical Problem

The disadvantages of therapeutic lenses known from the prior art are their inability to quickly absorb aggressive chemical agents that cause chemical burns to the eyes, as well as decay products resulting from damage of eye tissues. Thus, therapeutic lenses known from the prior art cannot be used to treat acute conditions caused by membrane damaging of the eye and in need of rapid relief of pain and removal of the damaging agent.

The disadvantage of lenses obtained on the basis of hydrogels with ion-exchange resins is their inability to induce regenerative processes for the rapid healing of a damaged organ of vision with the subsequent complete restoration of its functions. Moreover, there is the problem of frequent infections in the view of the inflammatory process in the eye.

In such lenses, due to the strong absorption capacity of the IER, it is impossible to use PAA together (sequentially) in view of their competitive absorption by IER. It is also impossible to use the method of saturation of the hydrogel by the drug, because such substances are also absorbed by ion exchangers.

### Solution to Problem

Therefore, the main course of this invention is to eliminate mentioned above disadvantages and to raise effectiveness of treatment.

The present invention solves the problem of eliminating the above disadvantages and obtaining hydrogels and lenses that eliminate these disadvantages, as well as providing a more effective combined approach in the treatment of acute chemical burns, injuries, severe infectious diseases.

This invention relates to a method of producing ion-exchange polymeric hydrogels for eye treatment, which includes monomer copolymerization under ionizing radiation in presence of linking agent and ionites, when copolymerization is carried out gradually to obtain a prepolymer of desired viscosity for filling lens forms, and ionites introduction in the form of finely dispersed powder, filing the hydrogel into lens forms and subsequent copolymerization till an adequate ionizing dosage is performed providing a gel suitable for lenses formation, characterized in that the gel is also filled with pharmaceutically active agent in the form of finely dispersed powder prior to ionite introduction, and the size of particles of PAA is lower that the size of ionite particles.

In this case, the introduction of PAA with lower particle size than IER, allows them to be introduced into a less viscous medium of monomers while maintaining homogeneous distribution. This introduction is maintained on earlier stage before final polymerization

### Advantageous Effects of Invention

Thus, the lenses obtained by the method according to the present invention have significant advantages over therapeutic and lenses for chemical injuries known in the art. They provide:
A full range of therapy measures throughout the course of treatment without changing the lenses, which ensures effectiveness and comfort, reduction of pain and the probability of penetration of an extraneous infection.
Accelerated removal of toxic substances and the early start of recovery processes.
Pain relief and greater comfort in the very initial stages of treatment.
Continuation of the removal of toxic products at later stages of treatment, including the decay products of diseased cells during therapy.

Moreover, the lenses of the present invention possess an ability of prolonged action, which provides the opportunity to replace less convenient methods of treatment, such as using eye drops, which require regular and frequent administration.

Also, lenses have improved physical characteristics, such as moisture content, due to the presence of components such as hyaluronic acid, making them more comfortable for wearing.

### DESCRIPTION OF EMBODIMENTS

In accordance with the invention, PAAs can be introduced both in the form of solid particles of small sizes, smaller than the particle sizes of IER, and in the form of nanocapsules, which are encapsulated drugs.

In accordance with the invention, PAAs can be introduced both in the form of solid particles of small sizes, smaller than the particle sizes of IER, and in the form of nanocapsules, which are encapsulated drugs.

Preliminary administration of PAA at the molecular level is also applicable. In this case, PAAs during subsequent irradiation can be “sewn” in the resulting gel, leaving their active endings exhibiting catalytic or other active healing properties free (for example, streptodornase, clostridium peptidase, hyaluronidase and other enzymes widely used in wound healing etc.)

In another aspect, the invention relates to the method of producing ion-exchange polymer hydrogels, characterized in that at the prepolymer stage PAA may be presented by microporous sorbents selected from the group consisting of silica gel, salts of alginic acids, carbon sorbents, zeolites in the form of fine powders.

In one another aspect the invention relates to the method of producing ion-exchange polymer hydrogels, characterized in that PAA is hyaluronic acid in the form of solid finely dispersed powder.

Hyaluronic acid (HA) is a polymer consisting from repeating units of D-glucuronic acid and N-acetyl-D-glucosamine, which found mainly in the extracellular space and joints. It's a very hydrophilic natural polymer with high biocompatibility and high moisture content (one molecule of HA can bind 200-500 molecules of water), due to what is widely used in medicine for treating of dry eye disease in the formulation of eye drops, for osteoarthritis therapy and for retaining skin moisture.

Recent studies have shown that HA also possesses anti-inflammatory and antibacterial activities. In addition, since free radicals can break down hyaluronic acid into smaller fragments in damaged tissues, it also has the antioxidant function of scavenging free radicals. Furthermore, it is well known that HA also can be a scaffold during tissue repair to provide cell climbing and migration opportunities. With these useful functions, HA is reported to accelerate regenerative processes. In yet another aspect, it has been shown that HA under some types of ionizing radiation breaks down into low-molecular-weight hyaluronic acid (LMWHA), which possesses high ability for wound healing (Yu-Chih Huang, Kuen-Yu Huang, Wei-Zhen Lew, Kang-Hsin Fan, Wei-Jen Chang, Haw-Ming Huang. Gamma-Irradiation-Prepared Low Molecular Weight Hyaluronic Acid Promotes Skin Wound Healing, 2019).

In another aspect, the invention relates to the method of producing ion-exchange polymer hydrogels, characterized in that PAA is taurine.

Taurine is a naturally occurring free amino acid, which is widely known for its neu-roprotective, antioxidant, cardiovascular and regenerative properties.

In ophthalmology taurine contributes to the improvement of energy processes. It stimulates reparative and regenerative processes in diseases of a dystrophic nature and/or diseases accompanied by a sharp violation of the metabolism of eye tissues. It contributes to the normalization of the functions of cell membranes, activation of energy metabolic processes. As an eye drop formulation it is widely used for corneal injury treatment (as a stimulant of reparative processes).

In physiological conditions (pH 7) taurine exists as the zwitterion H3N+CH2CH2SO3-, which is highly convenient for use in ion-exchange hydrogels since the molecule is not charged and is not absorbed by ionites.

In yet another aspect PAA represented by nanoparticles with antimicrobial function.

In the past few decades, tremendous interest and substantial research efforts were directed toward the biomedical evaluation and revaluation of metallic nanoparticles derived from noble metals, such as silver and gold, thanks to their specific and genuine chemical, biological, and physical properties. For example, AgNPs (Ag nanoparticles) were used for a long time as antibacterial agents in the health industry, cosmetics, food storage, textile coatings, and some environmental applications. The effectiveness of nanosilver-based biomaterials as promising antimicrobial agents was experimentally assessed against a wide range of medically relevant planktonic and sessile pathogenic microorganisms, including bacteria, viruses, fungi, and yeasts.

Thus, nanoparticles can be used as antibacterial agent, which also possesses ability for quick drug delivery to target tissues.

Whereas the nanoparicles have essentially low size, there are no strong requirements on viscosity and stability of their distribution in the gel until the end of copolymerization.

In another aspect the present invention relates to the method of producing ion-exchange hydrogels characterized in that PAA are introduced in capsules having a shell with a delayed PAA release.

Preferably the capsules have a shell consisting from alginic acid salts or chitosan.

Alginates, as well as hyaluronic acid, possess high water absorption properties, due to which prepared lenses could maintain desired humidity rate.

Sodium and calcium alginates are widely used in the art in the field of hydrogel formation. Due to its exclusive ability of gel formation almost any PAA could be included into capsules with alginate shell. Chitosan is a linear polysaccharide composed of randomly distributed beta-(1-4)-linked D-glucosamine(deacetylated unit) and N-acetyl-D-glucosamine (acetylated unit). In medicite it is used as antibacterial and haemostatic agent. Recently it has been shown that nanocapsules with chitosan shell can be used for drug delivery on the tissue cultures (Hydrogel nanoparticles of chitosan-folic acid conjugate with imatinib methanesulfonate/M. E. Lozovskaya, V. I. Kulikovskaya, Zh. V. Ignatovich, E. V. Koroleva, V. E. Agabekov//Pharmaceutical Chemistry Journal, 2018, Vol. 52, No. 2, P. 127-132).

The list of such pharmaceutically acceptable agents includes, but not limited by, taurine, retinol, cholecalciferol, tocopherol, lutein, zeaxanthin, flavonoids, antibiotics, anti-inflammatory and analgesic agents.

In another aspect the present invention relates to the method of producing ion-exchange hydrogels characterized in that above-mentioned capsules also includes nanoparticles or accelerated penetration of PAA throw biological membranes and ocular barriers.

Preferably nanoparticles included into capsules possess ferromagnetic or superparamagnetic properties for managed activation of PAA release.

It is more preferably to use superparamagnetic nanoparticles. In such particles the dipole moment of a single-domain particle fluctuates rapidly in the core due to the thermal excitation so that there is no magnetic moment for macroscopic time scales. Thus, these particles are non-magnetic when an external magnetic field is not applied but do develop a mean magnetic moment in an external magnetic field.

Advantages of the superparamagnetic particles are easy resuspension, large surface area, slow sedimentation and uniform distribution of the particles in the suspension media.

For example, in the nanocomplexes with chitosan and tripolyphosphate as a shell and cisplatin as a drug, ferromagnetic nanoparticles as magnetic nanoparticles with a diameter of 50 nm were used “Chemicell”, Germany). The drug was successfully released by a pulsed magnetic field using a low-intensity pulsed magnetic field apparatus (IMP) (ODO “Magomed”, Minsk, Republic of Belarus).

The exposure parameters were as follows: the amplitude of the magnetic field—1.3 T.; pulse duration—30 ms; exposure time—0.5 min.

Such exposure to magnetic pulses does not cause tissue heating, however, it ef-fectively implements the managed activation of PAA release.

However, a magnetotherapeutic effect is also present.

When using nanoparticles with magnetic properties in the composition of micro and nanocapsules with PAA, in addition to the main advantage of controlled drug release, there are additional possibilities for the controlled introduction of capsules with PAA into the hydrogel, both with regard to accelerated process of introducing capsules into the hydrogel under the influence of a constant magnetic field, and and also to the effective saturation of the hydrogel of the drug.

One more embodiment of this invention relates to therapeutic hydrogel contact lenses with ionites, produced by the above-mentioned method, wherein the mentioned above hydrogel includes bound and disturbed solid particles of PAA with sizes less that ionite particles size.

In another aspect these solid particles are capsules with PAA.

In yet another aspect PAA within the capsules is HA.

In another aspect capsules with PAA have a shell with delayed PAA release.

Preferably the shell is produced from alginates.

In yet another aspect capsules with PAA additionally contain magnetic and/or superparamagnetic nanoparticles for managed activation of PAA release.

Now therefore, the present invention relates to a method for producing ion-exchange hydrogels and lenses, produced from such hydrogel, which possess high ability to absorb acids, bases and other agents of undetermined nature, which cause chemical burns, as well as by-products of degenerative process, due to ionites. On the other side, such lenses possess prominent therapeutic properties due to introduction of pharmaceutically active agents in a form of finely dispersed powder for prolonged action.

Thus, new lenses produced in accordance with the present invention can provide an operational, continuous, both sequential and parallel treatment process of the wide range of eye damage with high efficiency and the least consequences.

The lenses of the present invention may also possess an analgesic effect.

Moreover, the lenses of the present invention have a prolonged action, which allows replacing with the method of the present invention less convenient methods of treatment, such as treatment with eye drops, which require regular and frequent administration.

Also, lenses possess improved physical characteristics, such as moisture content, due to the presence of components such as hyaluronic acid, making them more comfortable to wear.

Ionizing radiation are preferably includes gamma-radiation and UV-radiation.

A distinctive feature of the proposed method is that prior to the introduction of the powder of ion exchangers, the PAA in finely dispersed form is introduced into the prepared mixture of prepolymer. In this case, the particle size of the PAA should be smaller than the particle size of the ion exchangers, which prevents them from settling in the gel until the polymerization process is completed. Depending on the specific type of eye damage, various medicinal substances, sorbents, analgesics, healing, regenerative and other pharmaceutically active agents can be used.

In this case, solid drugs are preferably introduced in the form of a fine powder or in the composition of nanocapsules. Nanocapsules can be introduced directly into the monomers mixture, and finely dispersed PAA powder can be introduced into the prepolymer at an earlier stage of ionizing irradiation, which is sufficient for the formation of a gel of a certain viscosity, which ensures that the powder does not pre-cipitate until the copolymerization stage is completed.

Nanocapsules, when used, usually obtained and deposited in solutions or gels.

In such cases, PAA can be introduced with the solution, for example onto the surface of the monomer or prepolimer, followed by removal of the solvent, for example in vacuum. The same option can be used with macromolecular PAA.

### EXAMPLES

Prepolymer Production

1. A polymer composition is prepared using purified by double vacuum distillation N-vinyl pyrrolidone and methyl methacrylate, as well as diethylene glycol divinyl ether, in a ratio of 7:3:0.12 volume parts. Finely dispersed powder of the PAA is introduced into the mixture of the prepolymer prepared in accordance with the Example 1 to a final concentration of 0.1-0.3%. The prepared mixture is taken into 10 or 20 ml syringes. After that air is removed followed by tight closing of the syringes. The required number of them is prepared. The tubes are placed in a plastic bag, tightly closed and irradiated with ionized radiation until the absorbed dose in the amount of 3-5 kGy is achieved. The prepared prepolymer can be stored in the refrigerator for no more than 10 days at a temperature of 4.5±0.5 C.

2. Lenses with HA.

Prepolymer is prepared in accordance with example 1, with sodium salt of hyaluronic acid used as PAA. On the next stage, molds are prepared. Molds (puncheon and matrix) are cleaned with a clean, lint-free cloth dipped in rectified ethyl alcohol. The molds are assembled into three-position modules and filled through the matrix with an ion exchange resin of 0.008 g each. After filling the resin into the mold using a dispenser, 50-60 microl of prepolymer are poured. The holes of the molds are closed with a polyethylene gasket and pressed against the cover with a screw. Next, three-position molds are assembled into a polymerization module. The polymerization module then irradiated with ionizing radiation until the absorbed dose in the amount of 35 kGy is achieved. Then the molds are washed with an ultrasonic cleaner, wiped and dried. After polymerization, the module is removed and disassembled into molds. Molds together with the lenses are soaked in deionized water until the lenses completely swell and the lenses are out. On the next stage hydrated lenses are explored under the microscope and lenses with chips and bubbles are rejected. The selected lenses are then washed three times with deionized water using a shaker. The lenses are placed in bottles with deionized water and sterilized.

3. Lenses with Microporous Agents

The prepolymer is prepared in accordance with the Example 1. On next stage, microporous agents are added to the mixture to a final concentration of 0.01-0.15 microg/ml and slowly mixed until a homogeneous gel is achieved.

Then the finished polymer composition is used in the manufacturing of lenses according to the Example 2.

4. Lenses with Low-Molecular-Weight Hyaluronic Acid (LMWHA)

The lenses are made in accordance with the Example 2, while gamma-radiation up to an absorbed dose of 35 kGy is used as ionizing radiation.

5. Lenses with Taurine

The prepolymer is prepared in accordance with the Example 1. At the same time, fine taurine powder is added to the final dose of 40 mg/ml.

Then prepolymer composition is used in the manufacturing of lenses according to the Example 2.

6. Lenses with Nanoparticles

The prepolymer is prepared in accordance with the Example 1. At the next stage of gel formation nanoparticles are added in solid finely divided form in an amount from 30 to 100 ng. Then the finished polymer composition is used in the manufacturing of lenses according to Example 2.

7. Lenses Containing Nanocapsules with Alginate or Chitosan Shell, Wherein Such Capsules May Contain Different PAA (See Above)

The prepolymer is prepared in accordance with the Example 1. At the next stage of gel formation nanocapsules with PAA is added in the amount which is necessary for achieving desirable therapeutically effect. Then the finished polymer composition is used in the manufacturing of lenses according to Example 2.

8. Lenses Containing Capsules with Alginate Shell or Chitosan, Wherein Such Capsules Contain PAA and Nanoparticles for Quick Penetration

The lenses in prepared in accordance with the Example 6, wherein the above-mentioned nanocapsules also contain nanoparticles in the amount from 30 to 100 ng.

Lenses (as mentioned in ex. 6), wherein capsule shell includes ferromagnetic and su-perparamgnetic particles for managed release.

One skilled in the art will appreciate that other PAAs can be used in the present invention, as part of a lens, individually or in combination with other PAAs.

### INDUSTRIAL APPLICABILITY

Lenses obtained in accordance with the present invention can be used in medicine, namely for therapeutic use in ophthalmology for the treatment of chemical burn injuries. At the same time lenses possess anti-inflammatory, antibacterial and analgesic properties, as well as comfort wearing.

1. A method of producing ion-exchange polymeric hydrogels for eye treatment, which includes monomer copolymerization under ionizing radiation in presence of linking agent and ionites, when copolymerization is carried out gradually to obtain a prepolymer of desired viscosity for filling lens forms, and ionites introduction in the form of finely dispersed powder, filing the hydrogel into lens forms and subsequent copolymerization till an adequate ionizing dosage is performed providing a gel suitable for lenses formation, characterized in that the gel is also filled with pharmaceutically active agent in the form of finely dispersed powder prior to ionite introduction, and the size of particles of pharmaceutically active agent is lower that the size of ionite particles.

2. The method of producing ion-exchange polymer hydrogels according to claim 1, characterized in that at the prepolymer stage (during process) microporous sorbents selected from the group consisting of silica gel, salts of alginic acids, carbon sorbents, zeolites in the form of fine powders are additionally introduced.

3. The method of producing ion-exchange polymer hydrogels according to claim 1, characterized in that pharmaceutically active agent is hyaluronic acid in the form of solid finely dispersed powder.

4. The method of producing ion-exchange polymer hydrogels according to claim 3, characterized in that hyaluronic acid under some types of ionizing radiation breaks down into low-molecular-weight hyaluronic acid, which possesses high ability for wound healing.

5. The method of producing ion-exchange polymer hydrogels according to claim 1, characterized in that pharmaceutically active agent is taurine.

6. The method of producing ion-exchange polymer hydrogels according to claim 1, characterized in that pharmaceutically active agent represented by nanoparticles with antimicrobial function.

7. The method of producing ion-exchange hydrogels according to claim 1, characterized in that pharmaceutically active agent are introduced in capsules having a shell with a delayed pharmaceutically active agent release.

8. The method of producing ion-exchange hydrogels according to claim 7, characterized in that the capsules have a shell consisting from alginic acid salts or chitosan.

9. The method of producing ion-exchange hydrogels according to claim 7, characterized in that the pharmaceutically active agent introduced in capsules selected from a group consisting of taurine, retinol, cholecalciferol, tocopherol, lutein, zeaxanthin, flavonoids, antibiotics, anti-inflammatory and analgesic agents.

10. The method of producing ion-exchange hydrogels according to claim 7, characterized in that above-mentioned capsules also includes nanoparticles or accelerated penetration of pharmaceutically active agent throw ocular barriers.

11. The method of producing ion-exchange hydrogels according to claim 10, characterized in that nanoparticles included into capsules with pharmaceutically active agent, are antiferromagnetic or superparamagnetic nanoparticles for managed activation of pharmaceutically active agent release.

12. Therapeutic hydrogel contact lenses with ionites, produced from hydrogel in accordance with the claim 1, wherein the mentioned above hydrogel includes bound and distributed particles of pharmaceutically active agent with sizes less that ionite particles size.

13. Lenses according to claim 12, characterized in that particles are capsules with pharmaceutically active agent.

14. Lenses according to claim 12, characterized in that pharmaceutically active agent is hyaluronic acid.

15. Lenses according to claim 12, characterized in that particles are capsules with pharmaceutically active agent have a shell with delayed pharmaceutically active agent release.

16. Lenses according to claim 15, characterized in that capsules have shell produced from alginates or chitosan.

17. Lenses according to claim 12, characterized in that pharmaceutically active agent particle size range from 50 to 1000 nm.

18. Lenses according to claim 12, characterized in that pharmaceutically active agent particles includes magnetic and/or superparamagnetic nanoparticles.
